(19) United States
(12) Patent Application Publication
(10) Pub. No.: US 2023/0142728 A1
(43) Pub. Date: May 11, 2023

(54) LIVING BODY ABNORMALITY DETECTION DEVICE, LIVING BODY ABNORMALITY DETECTION METHOD, AND PROGRAM

(71) Applicant: DATA SOLUTIONS, INC., Tokyo (JP)

(72) Inventors: Tomoaki OTSUKI, Yokohama-shi (JP); Chen YE, Yokohama-shi (JP)

(73) Assignee: DATA SOLUTIONS, INC., Tokyo (JP)

(21) Appl. No.: 17/906,563

(22) PCT Filed: Mar. 11, 2021
(86) PCT No.: PCT/JP2021/009767

§ 371 Date: Sep. 16, 2022

(30) Foreign Application Priority Data
Mar. 17, 2020 (JP) 2020-046622

(51) Int. Cl. A61B 5/0245; A61B 5/00

(52) U.S. Cl. CPC A61B 5/0245; A61B 5/725; A61B 5/7267; A61B 5/746

(57) ABSTRACT

An object is to accurately detect abnormality of a living body. A living body abnormality detection device comprises: a signal acquirer that acquires a first signal including a frequency component of heartbeat; a filter that attenuates a frequency component higher than the frequency component of heartbeat and a frequency component lower than the frequency component of heartbeat based on the first signal to generate a second signal; a frequency analyzer that indicates an analysis result obtained by analyzing a frequency component of the second signal based on the second signal; an energy proportion calculator that calculates an energy proportion that is a proportion occupied by energy of a frequency component for each frequency band with respect to entire energy in the second signal based on the analysis result; a variance value calculator that calculates an energy variance value of a frequency component for each frequency band based on the analysis result; and a detector that at least detects abnormality or normality of a living body based on either one of the energy proportion and the variance value or both of the energy proportion and the variance value.

### TECHNICAL FIELD

The present invention relates to a living body abnormality detection device, a living body abnormality detection method, and a program.

### BACKGROUND ART

There is a known technique in which biological information such as a heart rate is measured with a wearable device and a notification is made to a user when there is an abnormality in the biological information (see Non-Patent Literature 1, for example).

In watching systems, observation equipment such as a nurse call button, a human detection sensor, a Doppler sensor, a heart rate monitor, a breath measurement device, a thermo camera, a sphygmomanometer, a clinical thermometer, an illuminometer, a thermometer, or a hygrometer is first connected to an observed person such as an elderly person. The watching system thus acquires observation information for the observed person. The watching system then determines whether or not an emergency notification condition is met based on the observation information, and makes an emergency notification in the case of an emergency. Watching systems that use such vital sensors are known (see Patent Literature 1, for example).

### CITATION LIST

### Non-Patent Literature

Non-Patent Literature 1: “Your heart rate. What it means, and where on Apple Watch (R) you'll find it.”, [online], Jan. 21, 2020, [retrieved on Mar. 2, 2020], Internet <URL: https://support.apple.com/ja-jp/HT204666>

### Patent Literature

Patent Literature 1: Japanese Patent Laid-Open No. 2017-151755

### SUMMARY OF INVENTION

### Technical Problem

In view of the fact that it is difficult for conventional techniques to accurately detect the abnormality of a living body, it is an object of the present invention to accurately detect the abnormality of a living body.

### Solution to Problem

A living body abnormality detection device is required to comprise:

a signal acquirer that acquires a first signal including a frequency component of heartbeat;

a filter that attenuates a frequency component higher than the frequency component of heartbeat and a frequency component lower than the frequency component of heartbeat based on the first signal to generate a second signal;

a frequency analyzer that indicates an analysis result obtained by analyzing a frequency component of the second signal based on the second signal;

an energy proportion calculator that calculates an energy proportion that is a proportion occupied by energy of a frequency component for each frequency band with respect to entire energy in the second signal based on the analysis result;

a variance value calculator that calculates an energy variance value of a frequency component for each frequency band based on the analysis result; and

a detector that at least detects abnormality or normality of a living body based on either one of the energy proportion and the variance value or both of the energy proportion and the variance value.

### Advantageous Effect of Invention

According to the disclosed technique, it is possible to accurately detect the abnormality of a living body.

### BRIEF DESCRIPTION OF DRAWINGS

FIG. 1 shows an example overall configuration of a first embodiment.

FIG. 2 shows an example of a Doppler radar.

FIG. 3 shows an example of a living body abnormality detection device.

FIG. 4 shows an example overall process of the first embodiment.

FIG. 5 shows an example of a first signal.

FIG. 6 shows an analysis result in an experiment in which abnormality occurs in a low band.

FIG. 7 shows an analysis result in an experiment in which no abnormality occurs in a living body.

FIG. 8 shows an analysis result in an experiment in which abnormality occurs in a high band.

FIG. 9 shows a result of an experiment of detecting abnormality.

FIG. 10 shows an example of a learning process.

FIG. 11 shows an example functional configuration.

FIG. 12 shows an example of IQ data measured by the Doppler radar.

### DESCRIPTION OF EMBODIMENTS

Optimal and minimal embodiments of the invention will be described below with reference to the drawings. Note that the same reference characters refer to similar components in the drawings, and overlapping descriptions will be omitted. Specific examples shown in the figures are illustrative, and further components other than those shown in the figures may also be included.

### First Embodiment

For example, a living body abnormality detection system 1 is a system with an overall configuration as described below.

<Example Overall Configuration>

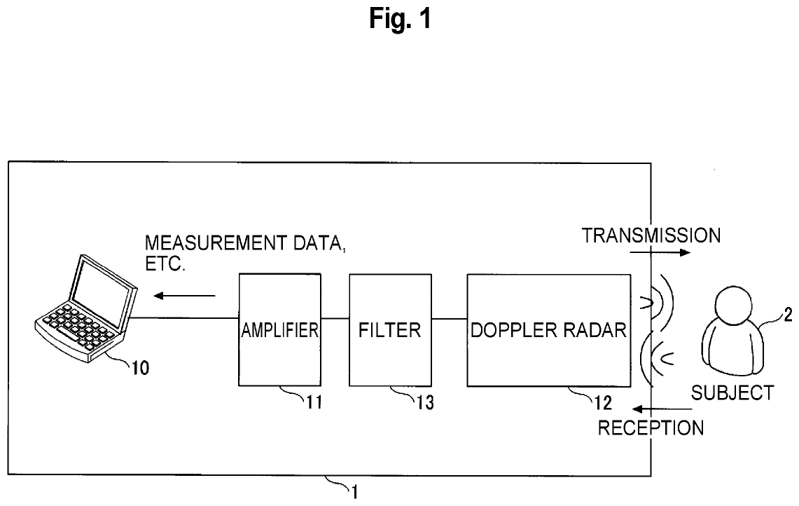

FIG. 1 shows an example overall configuration of a first embodiment. For example, the living body abnormality detection system 1 includes a personal computer (PC, hereinafter referred to as a “PC 10”), a Doppler radar 12, a filter 13 and the like. Note that the living body abnormality detection system 1 desirably includes an amplifier 11 or the like, as shown in the figure. The following description will be made with reference to the overall configuration shown in the figure by way of example.

The PC 10 is an information processing device and is an example of a living body abnormality detection device. The PC 10 is connected to peripheral devices such as the amplifier 11 via a network, a cable or the like. Note that the amplifier 11, the filter 13 and the like may be included in the PC 10. The amplifier 11, the filter 13 and the like may not be devices, but may be configured by software or configured by both hardware and software. The following description will be made with reference to the example of the living body abnormality detection system 1 as shown in the figure.

The Doppler radar 12 is an example of a measurement device.

In this example, the PC 10 is connected to the amplifier 11. The amplifier 11 is connected to the filter 13. The filter 13 is connected to the Doppler radar 12. The PC 10 acquires measurement data from the Doppler radar 12 via the amplifier 11 and the filter 13. That is, the measurement data is signal data indicating the action of a living body including heartbeat or the like. Next, the PC 10 analyzes the heartbeat or the like of the subject 2 based on the acquired measurement data, and measures the movement of the human body such as a heart rate.

The Doppler radar 12 acquires a signal (hereinafter referred to as a “biological signal”) indicating action such as heartbeat based on the following principle, for example.

<Example of Doppler Radar>

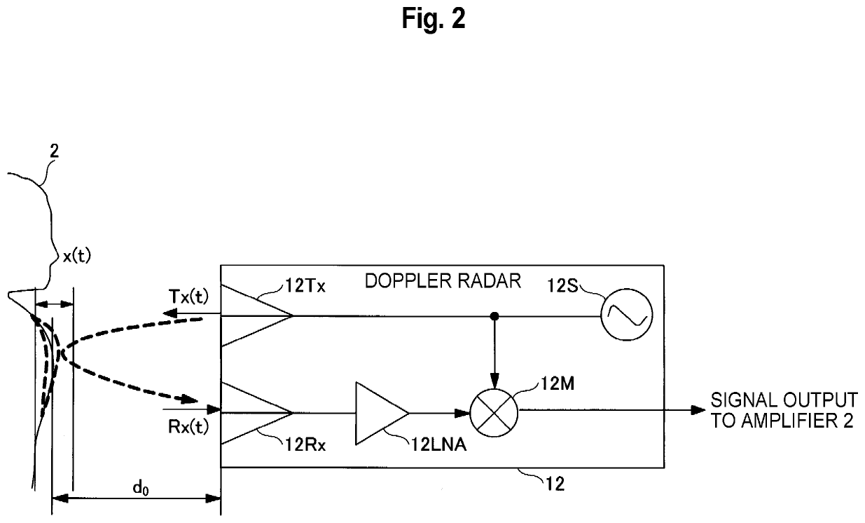

FIG. 2 shows an example of the Doppler radar. For example, the Doppler radar 12 is a device with a configuration as shown in FIG. 2. Specifically, the Doppler radar 12 includes a source 12S, a transmitter 12Tx, a receiver 12Rx, and a mixer 12M. The Doppler radar 12 also includes an adjuster 12LNA such as a low noise amplifier (LNA) for performing a process such as reducing the noise in data received by the receiver 12Rx.

The source 12S is a transmission source for generating a transmission wave signal transmitted by the transmitter 12Tx.

The transmitter 12Tx transmits the transmission wave to the subject 2. Note that the transmission wave signal can be represented by a function Tx(t) with respect to time “t”, and can be represented as in equation (1) below, for example.

[Expression 1]

Tx(t)=cos(ω_{c}t) (equation 1)

In equation (1) above, the letter “ω_{c}” represents the angular frequency of the transmission wave.

It is assumed that the subject 2, that is, the reflection surface of the transmitted signal has a displacement of x(t) at time “t”. In this example, the reflection surface is the chest wall of the subject 2. The displacement x(t) can be represented as in equation (2) below, for example.

[Expression 2]

x(t)=m×cos(ωt) (equation 2)

In equation (2) above, the letter “m” represents a constant indicating the amplitude of the displacement. Also, in equation (2) above, the letter “ω” represents the angular speed, which shifts due to the movement of the subject 2. Note that the variables similar to those in equation (1) above are the same variables.

The receiver 12Rx receives a reflected wave reflected by the subject 2 after being transmitted by the transmitter 12Tx. The reflected wave signal can be represented by a function Rx(t) with respect to time t, and can be represented as in equation (3) below, for example.

$$[ Expression 3 ]$$

$$Rx ( t ) = cos ( {ω}_{c} t - 2 π \cdot \frac{2 ( {d}_{0} + x ( t ) )}{λ} ) \qquad ( equation 3 )$$

In equation (3) above, the letter “do” represents the distance between the subject 2 and the Doppler radar 12. The letter “λ” represents the wavelength of the signal. The same notation applies hereinafter.

The Doppler radar 12 mixes the function Tx(t) (equation (1) above) indicating the transmission wave signal and the function R(t) (equation (3) above) indicating the reception wave signal to generate a Doppler signal. Note that the Doppler signal can be represented by a function B(t) with respect to time t, as in equation (4) below.

$$[ Expression 4 ]$$

$$B ( t ) = cos ( θ + 2 π \cdot \frac{2 x ( t )}{λ} ) \qquad ( equation 4 )$$

Defining the angular frequency of the Doppler signal as “ω_{d}”, the angular frequency ω_{d} of the Doppler signal can be represented as in equation (5) below.

$$[ Expression 5 ]$$

$${ω}_{d} = θ + 2 π \cdot \frac{2 x ( t )}{λ} \qquad ( equation 5 )$$

The phase “θ” in equation (4) above and equation (5) above can be represented as in equation (6) below.

$$[ Expression 6 ]$$

$$θ = 2 π \cdot \frac{2 {d}_{0}}{λ} + {θ}_{0} \qquad ( equation 6 )$$

In equation (6) above, the letter “θ₀” represents the phase shift at the chest wall of the subject 2, that is, at the reflection surface.

Next, the Doppler radar 12 outputs the position, speed or the like of the subject 2 based on the result of comparing the transmitted transmission wave signal and the received reception wave signal, that is, the result of calculation in the equations above.

For example, I-data (in-phase data) and Q-data (quadrature-phase data) can be generated from the reception wave. Then, the distance by which the chest wall of the subject 2 moves can be detected by using the I-data and Q-data. It is also possible to detect whether the chest wall of the subject 2 moves frontward or backward based on the phase indicated by the I-data and Q-data. Therefore, the movement of the chest wall due to heartbeat can detect an indicator of the heartbeat or the like by using changes in the frequencies of the transmission wave and reception wave.

<Example Hardware Configuration of Living Body Abnormality Detection Device>

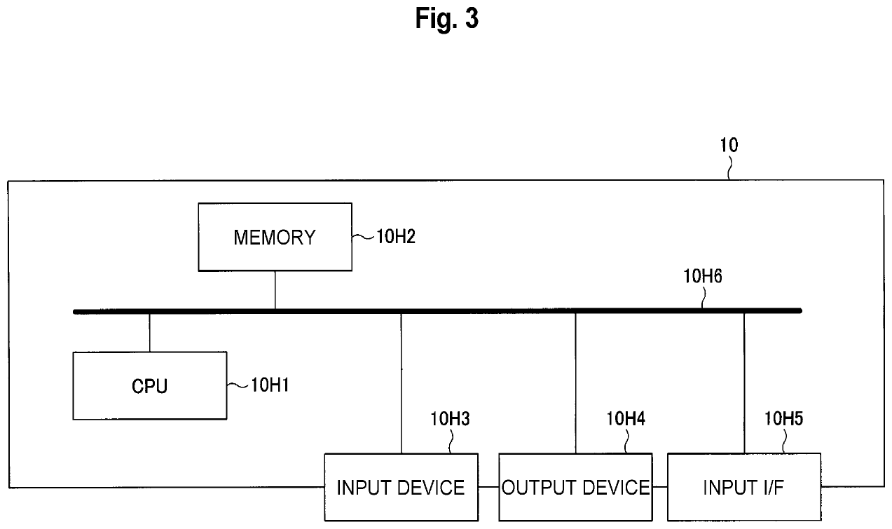

FIG. 3 shows an example of the living body abnormality detection device. For example, the PC 10 includes a central processing unit (CPU, hereinafter referred to as a “CPU 10H1”), a memory 10H2, an input device 10H3, an output device 10H4, and an input interface (I/F) (hereinafter referred to as an “input I/F 10H5”). Note that the hardware components included in the PC 10 are connected by a bus (hereinafter referred to as a “bus 10H6”), and data or the like is transmitted and received between the hardware components via the bus 10H6.

The CPU 10H1 is a control device for controlling the hardware components of the PC 10 and a computing device for performing computation for realizing various processing operations.

The memory 10H2 is a primary memory, an auxiliary memory and the like, for example. Specifically, the primary memory is a memory or the like, for example. The auxiliary memory is a hard disk or the like, for example. The memory 10H2 stores data including intermediate data used by the PC 10, programs used for various processing and control operations, and the like.

The input device 10H3 is a device for inputting parameters and instructions required for calculation to the PC 10 in response to an operation of the user. Specifically, the input device 10H3 is a keyboard, a mouse, a driver and the like, for example.

The output device 10H4 is a device for outputting various processing results and calculation results obtained by the PC 10 to the user or the like. Specifically, the output device 10H4 is a display or the like, for example.

The input I/F 10H5 is an interface connected to an external device such as a measurement device for transmitting and receiving data or the like. For example, the input I/F 10H5 is a connector, an antenna or the like. That is, the input I/F 10H5 transmits and receives data to/from the external device via a network, a wireless connection, a cable or the like.

Note that the hardware configuration is not limited to the configuration shown in the figure. For example, the PC 10 may further include a computing device, a memory or the like for performing processing in a parallel, distributed or redundant manner. The PC 10 may also be an information processing system connected to another device via a network or a cable for performing computation, control and storage in a parallel, distributed or redundant manner. That is, the present invention may be realized by an information processing system including one or more information processing devices.

The PC 10 thus acquires a biological signal indicating the action of the living body by using a measurement device such as the Doppler radar 12. Note that the biological signal may be acquired when necessary in real time, or may be collectively acquired by the PC 10 after a device such as the Doppler radar stores the biological signal for a certain period. A recording medium or the like may be used for the acquisition. The PC 10 may include a measurement device such as the Doppler radar 12, and the PC 10 may acquire the biological signal by performing measurement using the measurement device such as the Doppler radar 12 and generating the biological signal.

<Example Overall Process>

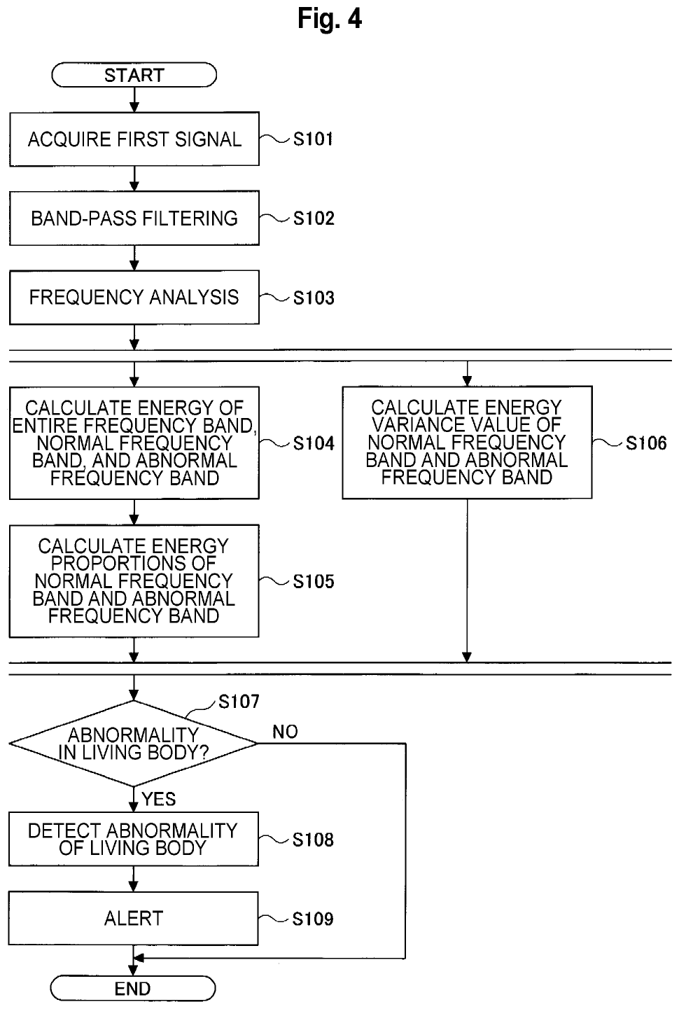

FIG. 4 shows an example overall process. For example, the overall process described below is performed every time window (preset to 60 seconds, for example).

(Example of Acquiring First Signal)

In step S101, the PC 10 acquires a first signal. For example, the first signal is a signal as shown below.

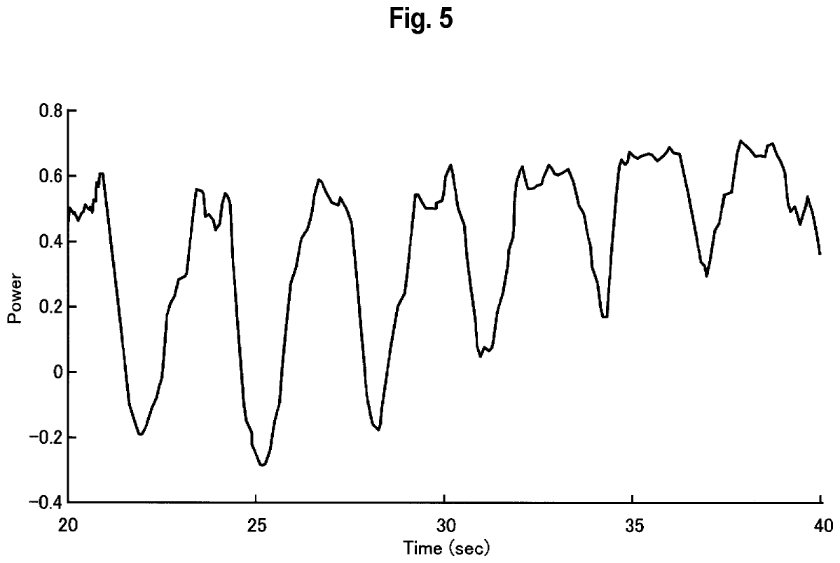

FIG. 5 shows an example of the first signal. In the figure, the horizontal axis indicates time, showing time points at which measurement is performed. The vertical axis indicates electric power estimated based on measurement results of the Doppler radar.

Hereinafter, a biological signal including a frequency component of heartbeat as shown in the figure is referred to as a “first signal”.

(Example of Band-Pass Filtering)

In step S102, the PC 10 performs band-pass filtering on the first signal to attenuate frequency components higher than the frequency component of heartbeat and frequency components lower than the frequency component of heartbeat. That is, the PC 10 attenuates frequency components of frequency bands other than the frequency component of heartbeat on the first signal. For example, the PC 10 performs filtering using a digital filter or the like with a cut-off frequency other than the frequency component of heartbeat.

For example, since the heart rate of an adult male is about 50 to 180 beats per minute, the frequency component of heartbeat mainly contains frequency components of about 0.8 Hz to 3 Hz. Therefore, to provide a margin such that the frequency component of heartbeat is not attenuated, the PC 10 desirably performs band-pass filtering to attenuate frequency components higher than 4.0 Hz and frequency components lower than 0.4 Hz. With such configuration, the PC 10 can attenuate frequency components that would be noise without attenuating the frequency component indicating heartbeat through the band-pass filtering.

Note that the frequency bands targeted by the band-pass filtering may be set in consideration of the age, sex, state and the like of the living body. For example, in a state of having done a heavy exercise or a state of being agitated, the heart rate has a higher frequency than in a resting state. Therefore, the frequency component of heartbeat is a frequency component higher than in the resting state. On the other hand, in the resting state, the frequency component of heartbeat is a low frequency component. Thus, in the PC 10, the frequency bands targeted by the band-pass filtering may be dynamically changed or narrowed down, for example, according to the state of the living body or the like.

Specifically, in a state in which it is considered that the frequency component of heartbeat is a high frequency component, such as a state of having done a heavy exercise, a heart rate of about 100 to 210 beats per minute (which corresponds to about 1.6 Hz to 3.5 Hz in frequency) is assumed, and the PC 10 performs band-pass filtering to attenuate other frequency components. On the other hand, in a state in which it is considered that the frequency component of heartbeat is a low frequency component, such as a resting state, a heart rate of about 50 to 84 beats per minute (which corresponds to about 0.8 Hz to 1.4 Hz in frequency) is assumed, and the PC 10 performs band-pass filtering to attenuate other frequency components.

As described above, a state or the like can be input or a value may be set in consideration of a state or the like to perform the band-pass filtering in accordance with the state.

Hereinafter, a signal generated by the band-pass filtering is referred to as a “second signal”.

(Example of Frequency Analysis)

In step S103, the PC 10 performs frequency analysis on the second signal. For example, the frequency analysis is realized by a fast Fourier transform (FFT) or the like. In this manner, the PC 10 calculates a spectrum indicating energy for each frequency band. It is desirable that the PC 10 indicates an analysis result in a normalized form and by a spectrum. Hereinafter, the spectrum is indicated by normalized values. A specific example of the analysis result will be described later.

The following description will be made with reference to an example in which a process of calculating an energy proportion (step S104 and step S105 in the figure) and a process of calculating an energy variance value (step S106 in the figure) are performed in parallel. However, these processes may not be parallel, but either one may be performed earlier.

(Example of Calculating Energy of Entire Frequency Band, Normal Frequency Band, and Abnormal Frequency Band)

In step S104, the PC 10 calculates energy of an entire frequency band, a normal frequency band, and an abnormal frequency band.

(Example of Calculating Energy Proportions of Normal Frequency Band and Abnormal Frequency Band)

In step S105, the PC 10 calculates energy proportions of the normal frequency band and the abnormal frequency band.

Note that the details of the energy and energy proportion of each frequency band calculated in step S104 and step S105 will be described later.

(Example of Calculating Energy Variance Values of Normal Frequency Band and Abnormal Frequency Band)

In step S106, the PC 10 calculates energy variance values of the normal frequency band and the abnormal frequency band.

The details of the energy variance values calculated in step S106 above will be described later.

(Example of Determining Whether or not Living Body is Abnormal Based on Either One of Energy Proportion and Variance Value or Both of Energy Proportion and Variance Value)

In step S107, the PC 10 determines whether or not the living body is abnormal based on either one of the energy proportion and the variance value or both of the energy proportion and the variance value.

Next, if it is determined that there is abnormality in the living body (YES in step S107), the PC 10 proceeds to step S108. On the other hand, if it is determined that there is no abnormality in the living body (NO in step S107), the PC 10 ends the overall process.

(Example of Detecting Abnormality of Living Body)

In step S108, the PC 10 detects abnormality of the living body.

If abnormality of the living body is detected in step S107 or step S108 shown above, the PC 10 desirably provides an alert as described below.

(Example of Providing Alert)

In step S109, the PC 10 provides an alert.

For example, the alert is a message or the like informing the user or a predetermined recipient that abnormality occurs in the living body. Therefore, the alert may be in any form as long as it can inform the user or the recipient of the abnormality. For example, the alert may be provided by light, sound, a notification of the heart rate, a message with predetermined text, or a combination thereof. Providing an alert in this manner can quickly inform that abnormality occurs in the living body.

<Experimental Result>

For example, the following analysis result is obtained as the analysis result of the frequency analysis, that is, step S103 by experiments.

<Example of Analysis Result of Frequency Analysis>

Hereinafter, a spectrum indicating frequency components on the horizontal axis and energy for each frequency component on the vertical axis is indicated by normalized values.

In the following analysis result, the entire frequency band considered, R1, corresponds to 30 bpm (beats per minute, a unit indicating the heart rate per minute) to 180 bpm. Therefore, when converted into frequency, the entire frequency band R1 is a frequency band of “30 bpm÷60 sec=0.5 Hz” to “180 bpm÷60 sec=3.0 Hz”. Thus, the entire frequency band R1 may be set to a certain limited range such as “0.5 Hz” to “3.0 Hz” as long as it is within the range of a frequency band obtained from the living body such as 0.5 Hz to 3.5 Hz.

In this experiment, a normal frequency band R2 corresponds to 50 bpm to 120 bpm. Thus, it is desirable that the frequency band of “normality” is configurable. Therefore, when converted into frequency, the normal frequency band R2 is a frequency band of “50 bpm÷60 sec=0.83 . . . Hz ≈0.83 Hz” to “120 bpm÷60 sec=2.0 Hz”.

A frequency band other than the normal frequency band R2 in the entire frequency band R1 is defined as an abnormal frequency band. Hereinafter, an abnormal frequency band in a frequency band lower than the normal frequency band R2 is simply referred to as a “low band R3”. An abnormal frequency band in a frequency band higher than the normal frequency band R2 is simply referred to as a “high band R4”.

When converted it into frequency, the low band R3 is a frequency band of “30 bpm÷60 sec=0.5 Hz” to “50 bpm÷60 sec=0.83 . . . Hz≈0.83 Hz”.

When converted into frequency, the high band R4 is a frequency band of “120 bpm÷60 sec=2.0 Hz” to “180 bpm÷60 sec=3.0 Hz”.

Thus, it is desirable that abnormality is classified by dividing the abnormal frequency band into the low band R3 and the high band R4. The following description will be made with reference to an example of using classification into three, “normal”, “high band”, and “low band”. However, the normal frequency band may be classified into “high”, “middle”, “low”, and the like. In addition, the classification may be performed by further dividing the frequency bands into smaller frequency bands. Further, the classification may be classification into two, “normal” and “abnormal”.

<Experimental Result Obtained When Abnormality Occurs in Low Band>

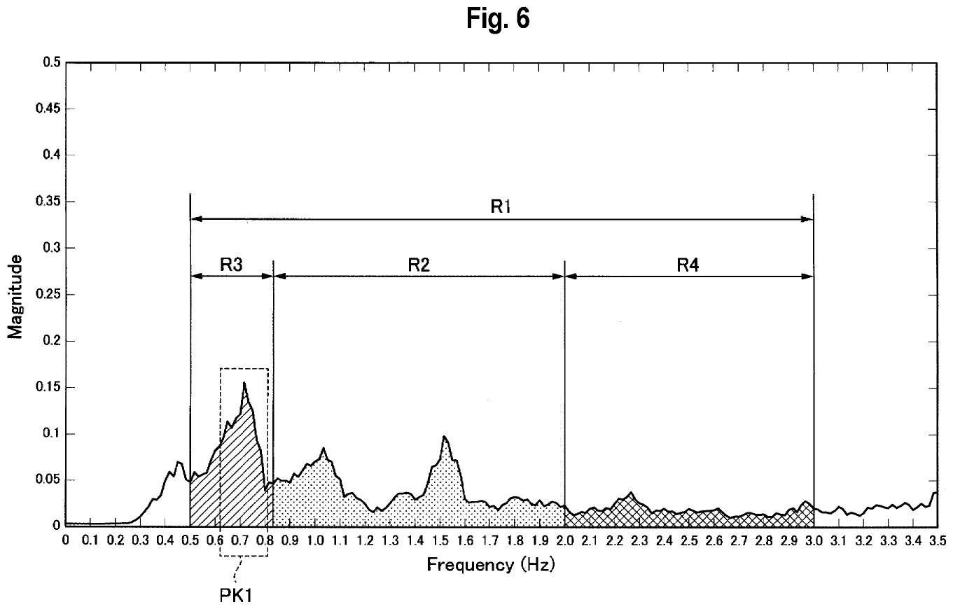

FIG. 6 shows an analysis result in an experiment in which abnormality occurs in the low band. This case is a case where abnormality in which the heart rate of the living body is low at “45.7 bpm” occurs. Thus, energy in the low band R3 is relatively high, as indicated by a first peak PK1. In this experiment, the energy proportion of the normal frequency band R2, the energy proportion of the low band R3, and the energy proportion of the high band R4, that is, calculation results of step S105 are the following values.

The energy proportion of the low band R3 is “30.7%”.

The energy proportion of the normal frequency band R2 is “49.8%”.

The energy proportion of the high band R4 is “19.5%”.

In this experiment, the variance value of the normal frequency band R2, the variance value of the low band R3, and the variance value of the high band R4, that is, calculation results of step S106 are the following values.

The variance value of the low band R3 is “3556.7×10⁻⁶”.

The variance value of the normal frequency band R2 is “918.8×10⁻⁶”.

The variance value of the high band R4 is “118.1×10⁻⁶”.

<Experimental Result Obtained When No Abnormality Occurs in Living Body>

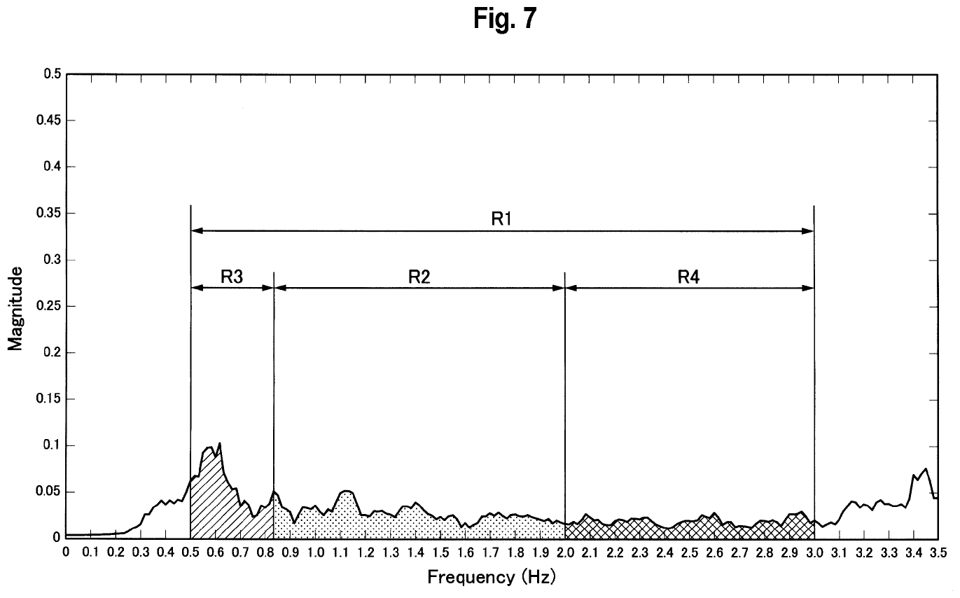

FIG. 7 shows an analysis result in an experiment in which no abnormality occurs in the living body. In this case, the heart rate of the living body is normal at “67.7 bpm”, and the frequency component of heart rate is in a “normal” state. Thus, a peak is indistinctive in the result, as compared to when abnormality occurs.

The energy proportions, that is, calculation results of step S105, calculated in a manner similar to the case of abnormality, are the following values.

The energy proportion of the low band R3 is “28.1%”.

The energy proportion of the normal frequency band R2 is “45.1%”.

The energy proportion of the high band R4 is “26.8%”.

The variance values, that is, calculation results of step S106, calculated in a manner similar to the case of abnormality, are the following values.

The variance value of the low band R3 is “1820×10⁻⁶”.

The variance value of the normal frequency band R2 is “272.2×10⁻⁶”.

The variance value of the high band R4 is “114.3×10^{-6”.}

<Experimental Result Obtained when Abnormality Occurs in High Band>

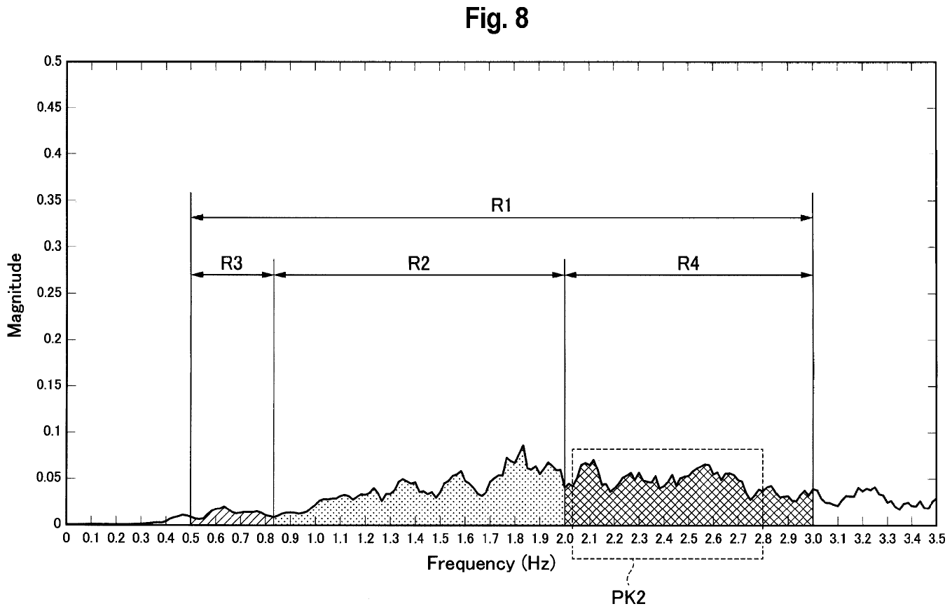

FIG. 8 shows an analysis result in an experiment in which abnormality occurs in the high band. This case is a case where abnormality in which the heart rate of the living body is high at “123.5 bpm” occurs. Thus, energy in the high band R4 is high, as indicated by a second peak PK2.

The energy proportions, that is, calculation results of step S105, calculated in a manner similar to other cases, are the following values.

The energy proportion of the low band R3 is “4.5%”.

The energy proportion of the normal frequency band R2 is “47.9%”.

The energy proportion of the high band R4 is “47.6%”.

The variance values, that is, calculation results of step S106, calculated in a manner similar to other cases, are the following values.

The variance value of the low band R3 is “59.9×10⁻⁶”.

The variance value of the normal frequency band R2 is “765.0×10⁻⁶”.

The variance value of the high band R4 is “596.5×10⁻⁶”.

Energy is calculated by integrating the frequency bands (resulting in the surface area of the frequency bands in the figure). Therefore, the respective energy proportions are calculated by calculating the entire energy and calculating proportions occupied by energy of the respective frequency bands.

As described above, when abnormality occurs, the variance value and the energy proportion of the abnormal frequency band are higher values than in the case of “normality”. Therefore, the PC 10 detects that abnormality occurs in the living body when either one of the variance value and the energy proportion is a high value. Thus, abnormality may be detected in a configuration in which it is determined on the whole that there is abnormality when either one of the variance value and the energy proportion is a high value, that is, in an “OR” configuration.

However, the PC 10 desirably has a configuration in which abnormality is detected on the whole when abnormality of the living body is detected in both determinations for the variance value and the energy proportion, that is, an “AND” configuration.

That is, the PC 10 first determines whether or not the living body is abnormal separately based on the variance value and the energy proportion. Next, the PC 10 detects abnormality of the living body in the case of a detection result that the living body is abnormal as it is determined that the values are high in both determination results (YES in step S107 and step S108).

Thus, the PC 10 is desirably configured to use the “AND” of both determinations for the variance value and the energy proportion. With such an “AND” configuration, the PC 10 can accurately determine abnormality of the living body.

<Result of Detection of Abnormality>

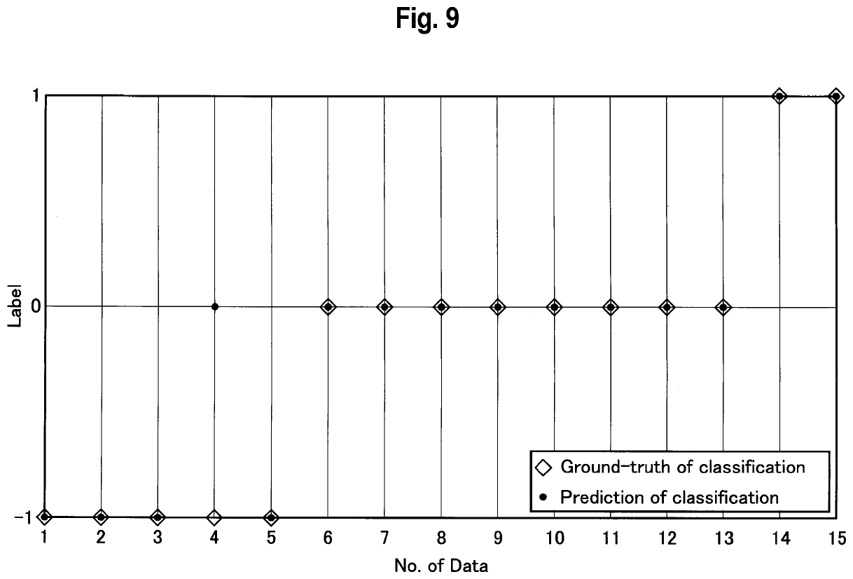

FIG. 9 shows a result of an experiment of detecting abnormality. The horizontal axis in the figure indicates the serial numbers of experimental results. On the vertical axis, “0” indicates a detection result of “normality”. Also, on the vertical axis, “−1” indicates a detection result of “abnormality of a low heart rate”. Also, on the vertical axis, “1” indicates a detection result of “abnormality of a high heart rate”. Therefore, coincidence on the vertical axis between a true value indicated by “Ground-truth of classification” and a detection result of “Prediction of classification”, which is a detection result of this embodiment, means a result in which abnormality is accurately detected.

As shown in the figure, in the experimental results other than “4”, the detection results for abnormality of a low heart rate, a normal heart rate, and abnormality of a high heart rate coincide. Thus, the experimental results show that the PC 10 can accurately detect abnormality and can classify the types of abnormality.

If abnormality is detected when the energy variance value and the energy proportion in the abnormal frequency band are high, as described above, it is possible to accurately detect abnormality of the living body.

Note that whether or not the variance value and the energy proportion are high values is determined by comparison to a preset threshold, for example. Note that the threshold is set in consideration of a result of an experiment performed in advance, such as the above-described experiment. The criteria for the energy and the variance value often vary according to the normalization method and the living body.

The abnormal frequency band and the threshold may be changed according to the state of the living body. For example, after doing a heavy exercise or the like, there is often no abnormality even if the heart rate is about “100 bpm” or more. On the other hand, if the heart rate is about “100 bpm” or more in the resting state, it may be determined that there is abnormality. Thus, the ranges of “normality” and “abnormality” vary according to conditions such as the state, age, sex, or mental state of the living body, or a combination thereof. Therefore, the abnormal frequency band, the threshold and the like may be changed according to these conditions.

### Second Embodiment

As compared to the first embodiment, a second embodiment has a configuration of using machine learning for the detection of abnormality. Hereinafter, the difference from the first embodiment will be mainly described, and overlapping descriptions will be omitted.

In the second embodiment, it is desirable that a learning process as described below is performed before the process shown in FIG. 4 is performed.

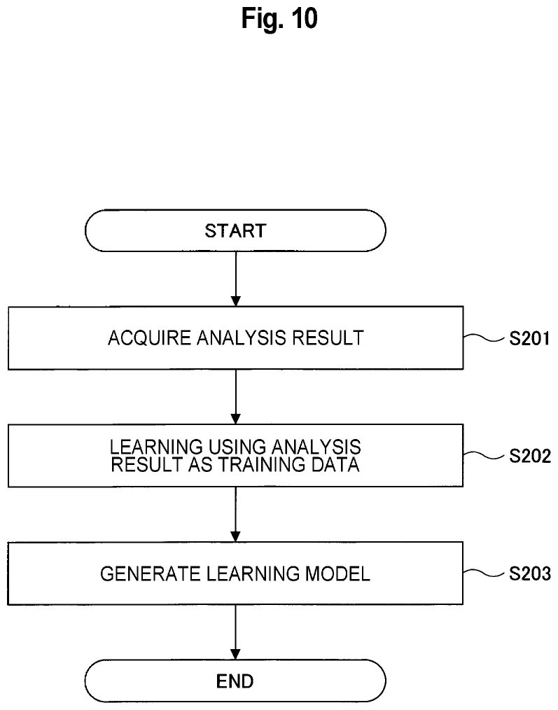

FIG. 10 shows an example of the learning process. That is, defining the overall process shown in FIG. 4 as an “execution process”, the PC 10 learns a learning model and generates a “learned model” through the learning process as shown in the figure before performing the “execution process”.

(Example of Acquisition of Analysis Result)

In step S201, the PC 10 acquires an analysis result of frequency analysis. For example, the PC 10 acquires data indicating an analysis result of frequency analysis obtained by performing processes similar to step S101 to step S103 in the first embodiment.

(Example of Learning Using Analysis Result as Training Data)

In step S202, the PC 10 learns a learning model by using the analysis result acquired in step S201 as training data. Note that the learning is desirably performed repeatedly according to the accuracy of detecting abnormality to an extent that the accuracy is obtained.

(Example of Generating Learned Model)

In step S203, the PC 10 generates a learned model.

For example, the learning model is desirably a support vector machine (SVM). That is, it is desirable that SVM learning is performed by using the energy proportion and the variance value as feature values to generate the learned model.

As shown in the first embodiment, the PC 10 detects abnormality of the living body by classifying the state of the living body into “abnormality” and “normality”. In addition, for example, even in the case of “abnormality”, it is desirable that the type of “abnormality” can be further classified, such as whether it is abnormality in the “low band” or abnormality in the “high band”. That is, the threshold for classification is learned by machine learning. Thus, by using an SVM learned model, it is possible to accurately classify the state of the living body.

The living body abnormality detection device and the living body abnormality detection system may be configured to use other artificial intelligence (AI). For example, the learned model may be a network structure including a network structure such as a convolution neural network (CNN) or a recurrent neural network (RNN). For example, the learning model is subjected to machine learning using image data indicating the analysis result of frequency analysis such as in FIG. 6 as training data. With such a configuration, the extraction of feature values can be eliminated.

Note that the training data may be in the form of a biological signal, image data indicating the analysis result of frequency analysis such as in FIG. 6, a numerical value such as the energy proportion, or a combination thereof.

The learned model is used as part of software in the AI. Therefore, the learned model is a program. Thus, the learned model may be distributed or executed via a recording medium, a network or the like, for example. In the execution process, the detection of abnormality is performed by using the learned model.

Note that the “learning process” and the “execution process” may be performed by different devices. Therefore, a device for performing the “learning process” may have a functional configuration that does not include a configuration for the “execution process”. On the other hand, a device for performing the “execution process” may have a functional configuration that does not include a configuration for the “learning process”. That is, the living body abnormality detection device and the living body abnormality detection system may have a functional configuration including either one of the configurations for the “learning process” and the “execution process”, not both.

### Third Embodiment

As compared to the first embodiment, the third embodiment has a difference in that a temporal difference of signal values indicated by the second signal is calculated. Hereinafter, the difference from the first embodiment and the like will be mainly described, and overlapping descriptions will be omitted.

For example, it is assumed that the second signal value is a signal value “X” shown in equation (7) below.

[Expression 7]

X=[x₁, x₂, x₃, . . . , xₙ₋₂, x_{n'11}, xₙ] (equation 7)

As indicated by equation (7) above, the signal value “X” is a value indicated by the second signal value at a certain time point. Also, “n” in equation (7) above is a value indicating the sequence number at which the signal value is acquired.

For example, the temporal difference is the difference between a signal value (hereinafter referred to as a “first signal value”) at a time point of “n” (hereinafter referred to as a “first time point”) and a signal value (hereinafter referred to as a “second signal value”) at a time point of “n−1” (hereinafter referred to as a “second time point”). Specifically, as indicated by equation (8) below, the temporal difference, “D”, is a result obtained by calculating the difference between the first signal value and the second signal value acquired at the second time point, which is the next previous time point to the first time point (indicated as “Xₙ”-“Xₙ₋₁” in equation (8) below).

[Expression 8]

D=[Xₙ−Xₙ₋₁]=[x₂−x₁, x₃−x₂, . . . , xₙ₋₂−xₙ₋₁, xₙ₋₁−xₙ] (equation 8)

As in equation (8) above, a temporal difference of signal values indicated by the second signal, that is, a signal obtained by performing band-pass filtering (step S102) on a biological signal is calculated. Note that, although a difference is calculated in equation (8) above for execution by a computer or the like, differentiation may be used for continuity.

In the frequency analysis in step S103, the PC 10 performs the analysis on the calculation result of the temporal difference, that is, the calculation result of equation (8) above.

As described above, the PC 10 is desirably configured to calculate the temporal difference. With such a configuration, the PC 10 can accurately detect abnormality.

<Example Functional Configuration>

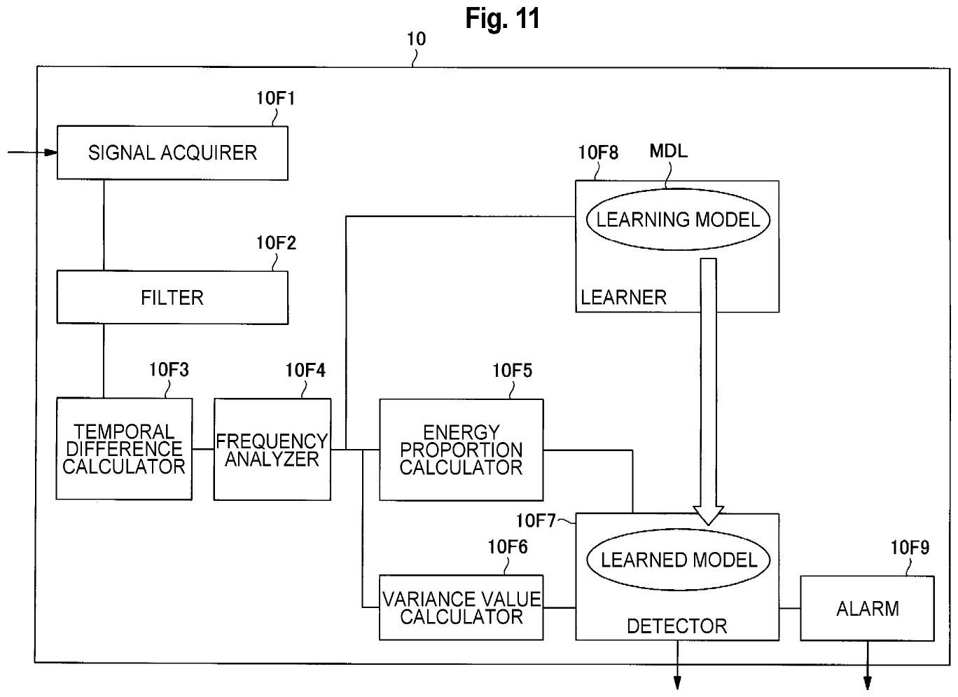

FIG. 11 shows an example functional configuration. For example, the living body abnormality detection device has a functional configuration including a signal acquirer 10F1, a filter 10F2, a frequency analyzer 10F4, an energy proportion calculator 10F5, a variance value calculator 10F6, and a detector 10F7. In addition, the living body abnormality detection device desirably has a functional configuration further including a temporal difference calculator 10F3, a learner 10F8, and an alarm 10F9 as shown in the figure. The following description will be made with reference to the functional configuration as shown in the figure by way of example.

The signal acquirer 10F1 performs a signal acquisition procedure of acquiring a biological signal such as the first signal. For example, the signal acquirer 10F1 is realized by the Doppler radar 12, the input I/F 10H5 or the like.

The filter 10F2 performs a filter procedure of filtering a certain frequency band in the biological signal such as the first signal. For example, the filter 10F2 is realized by the CPU 10H1, the filter 13 or the like.

The temporal difference calculator 10F3 performs a temporal difference calculation procedure of calculating a temporal difference based on the second signal. For example, the temporal difference calculator 10F3 is realized by the CPU 10H1 or the like.

The frequency analyzer 10F4 performs a frequency analysis procedure of performing frequency analysis on the second signal or the like or the temporal difference. For example, the frequency analyzer 10F4 is realized by the CPU 10H1 or the like.

The energy proportion calculator 10F5 performs an energy proportion calculation procedure of calculating an energy proportion based on the result of analysis by the frequency analyzer 10F4. For example, the energy proportion calculator 10F5 is realized by the CPU 10H1 or the like.

The variance value calculator 10F6 performs a variance value calculation procedure of calculating a variance value based on the result of analysis by the frequency analyzer 10F4. For example, the variance value calculator 10F6 is realized by the CPU 10H1 or the like.

The detector 10F7 performs a detection procedure of detecting abnormality of the living body based on either one of the energy proportion and the variance value or both of the energy proportion and the variance value. For example, the detector 10F7 is realized by the CPU 10H1 or the like.

The learner 10F8 performs learning procedure of learning a learning model MDL by using data or the like indicating the result of analysis by the frequency analyzer 10F4 as training data to generate a learned model. For example, the learner 10F8 is realized by the CPU 10H1 or the like.

The alarm 10F9 performs an alert procedure of providing an alert when abnormality occurs in the living body based on the result of detection by the detector 10F7. For example, the alarm 10F9 is realized by the output device 10H4 or the like.

<Example of IQ Data Measured by Doppler Radar>

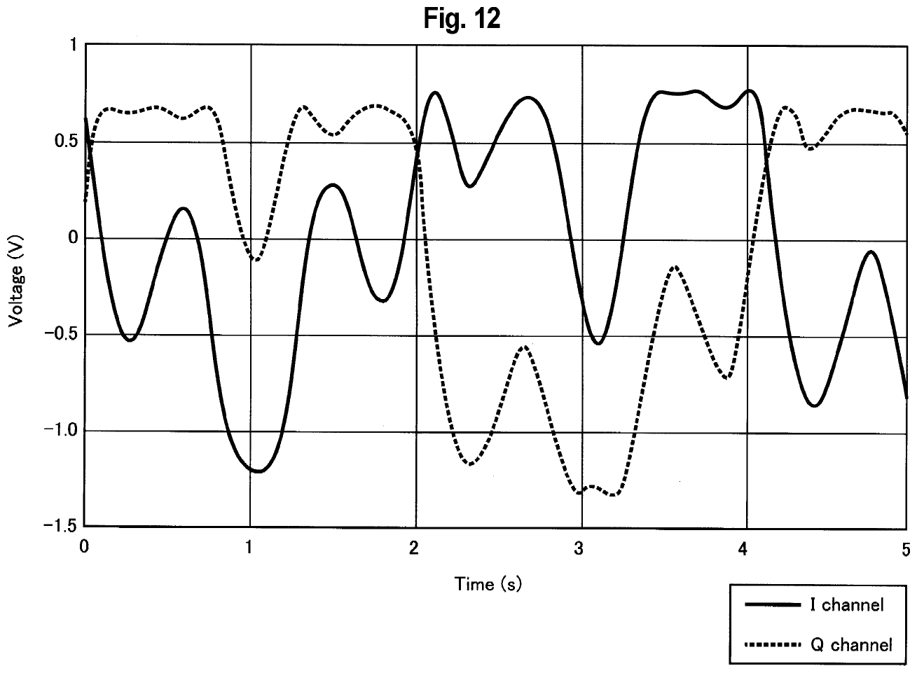

FIG. 12 shows an example of IQ data measured by the Doppler radar. For example, the Doppler radar 12 outputs a signal as shown in the figure. The arctan (Q/I) is then calculated to obtain a biological signal.

The Doppler radar 12 can measure the movement of an object based on the Doppler effect, by which the frequency of reflected waves changes when a moving object is irradiated with radio waves. Such a configuration that can measure the movement of a subject in a contactless manner is desirable.

<Variation>

Note that energy distribution in a region in which heartbeat is present possibly varies temporally. Therefore, the energy, the energy proportion and the like may be dynamically calculated according to the temporal variation of the energy distribution. In particular, under the condition that the time width is beyond an extent that the heart rate changes and a change of energy due to the environment is not large as compared to the change of heartbeat for the time width, it is desirable that the temporal variation is taken into consideration.

Note that the living body is not limited to a human but may be an animal or the like.

In addition, the biological signal may include breathing. Therefore, the abnormality detection method may also be performed by using the breathing rate, the frequency of breathing and the like. Note that, in the case of using breathing, it often differs in the number of counts per unit time from the heart rate, and therefore the threshold for detection, the range for determining abnormality, the range for determining normality and the like are desirably set separately for the breathing rate.

### Other Embodiments

For example, a transmitter, a receiver, or an information processing device may be a plurality of devices. That is, processing and control may be performed in a virtualized, parallel, distributed or redundant manner. On the other hand, the transmitter, receiver and information processing device may be integrated in hardware or share devices.

Note that all or part of each process according to the present invention may be written in a low-level language such as assembler or a high-level language such as an object-oriented language and realized by a program for causing a computer to perform the living body abnormality detection method. That is, the program is a computer program for causing a computer of the information processing device, the living body abnormality detection system or the like to perform each process.

Therefore, when each process is performed based on the program, a computing device and a control device included in the computer perform computation and control based on the program in order to perform each process. In order to perform each process, a memory included in the computer stores data used for the process based on the program.

The program can be recorded on a computer-readable recording medium and distributed. Note that the recording medium is a medium such as a magnetic tape, a flash memory, an optical disk, a magneto-optical disk or a magnetic disk. The program can be distributed through telecommunication lines.

Although preferred embodiments and the like have been described in detail above, there is no limitation to the above-described embodiments and the like, and various modifications and replacements can be made to the above-described embodiments and the like without departing from the scope of the claims.

This international application claims priority based on Japanese Patent Application No. 2020-046622, filed on Mar. 17, 2020, the entire contents of which are hereby incorporated by reference into this international application.

### REFERENCE SIGNS LIST

1: living body abnormality detection system
2: subject
10: PC
10F1: signal acquirer
10F2: filter
10F3: temporal difference calculator
10F4: frequency analyzer
10F5: energy proportion calculator
10F6: variance value calculator
10F7: detector
10F8: learner
10F9: alarm
11: amplifier
12: Doppler radar
13: filter
MDL: learning model
PK1: first peak
PK2: second peak
R1: entire frequency band
R2: normal frequency band
R3: low band
R4: high band

1. A living body abnormality detection device comprising:
a signal acquirer that acquires a first signal including a frequency component of heartbeat;
a filter that attenuates a frequency component higher than the frequency component of heartbeat and a frequency component lower than the frequency component of heartbeat based on the first signal to generate a second signal;
a frequency analyzer that indicates an analysis result obtained by analyzing a frequency component of the second signal based on the second signal;
an energy proportion calculator that calculates an energy proportion that is a proportion occupied by energy of a frequency component for each frequency band with respect to entire energy in the second signal based on the analysis result;
a variance value calculator that calculates an energy variance value of a frequency component for each frequency band based on the analysis result; and
a detector that at least detects abnormality or normality of a living body based on either one of the energy proportion and the variance value or both of the energy proportion and the variance value.

2. The living body abnormality detection device according to claim 1, wherein
the signal acquirer acquires the first signal by means of a Doppler radar.

3. The living body abnormality detection device according to claim 1, wherein
the filter performs band-pass filtering to attenuate a frequency component higher than 4.0 Hz and a frequency component lower than 0.4 Hz.

4. The living body abnormality detection device according to claim 1, further comprising:
a temporal difference calculator that calculates a temporal difference that is a difference between a first signal value at a first time point indicated by the second signal and a second signal value at a second time point different from the first time point based on the second signal, wherein
the frequency analyzer indicates the analysis result obtained by analyzing a frequency component of the temporal difference.

5. The living body abnormality detection device according to claim 1, wherein
the frequency analyzer analyzes a frequency band of 0.5 Hz to 3.5 Hz as an entire frequency band,
a frequency band of 0.83 Hz to 2.0 Hz in the entire frequency band is a normal frequency band, and
a frequency band lower than the normal frequency band and a frequency band higher than the normal frequency band in the entire frequency band are abnormal frequency bands.

6. The living body abnormality detection device according to claim 1, wherein
the detector detects abnormality of the living body based on a learned model generated by performing learning by using data indicating the analysis result as training data.

7. The living body abnormality detection device according to claim 6, wherein
the learned model is generated by learning an SVM learning model.

8. The living body abnormality detection device according to claim 1, further comprising:
an alarm that provides an alert when abnormality occurs in the living body based on a result of detection by the detector.

9. The living body abnormality detection device according to claim 1, wherein
the detector detects abnormality of the living body when, based on both of the energy proportion and the variance value, it is determined that there is abnormality in both determinations.

10. A living body abnormality detection method performed by a living body abnormality detection device, the living body abnormality detection method comprising:
a signal acquisition procedure in which the living body abnormality detection device acquires a first signal including a frequency component of heartbeat;
a filter procedure in which the living body abnormality detection device attenuates a frequency component higher than the frequency component of heartbeat and a frequency component lower than the frequency component of heartbeat based on the first signal to generate a second signal;
a frequency analysis procedure in which the living body abnormality detection device indicates an analysis result obtained by analyzing a frequency component of the second signal based on the second signal;
an energy proportion calculation procedure in which the living body abnormality detection device calculates an energy proportion that is a proportion occupied by energy of a frequency component for each frequency band with respect to entire energy in the second signal based on the analysis result;
a variance value calculation procedure in which the living body abnormality detection device calculates an energy variance value of a frequency component for each frequency band based on the analysis result; and
a detection procedure in which the living body abnormality detection device at least detects abnormality or normality of a living body based on either one of the energy proportion and the variance value or both of the energy proportion and the variance value.

11. A program for causing a computer to perform the living body abnormality detection method according to claim 10.
